(19) United States
(12) Patent Application Publication
(10) Pub. No.: US 2022/0048847 A1
(43) Pub. Date: Feb. 17, 2022

(54) PROCESS FOR PREPARING SUBSTITUTED ANILINES

(71) Applicant: Bayer Aktiengesellschaft, Leverkusen (DE)

(72) Inventors: Andreas REMBIAK, Bad Soden (DE); Florian ERVER, Wiesbaden (DE); Günter HÖMBERGER, Eppstein (DE)

(73) Assignee: Bayer Aktiengesellschaft, Leverkusen (DE)

(21) Appl. No.: 17/309,617

(22) PCT Filed: Dec. 12, 2019
(86) PCT No.: PCT/EP2019/084881

§ 371 Date: Jun. 10, 2021

(30) Foreign Application Priority Data
Dec. 20, 2018 (EP) 18214440.2

(51) Int. Cl. C07C 209/74

(52) U.S. Cl. CPC C07C 209/74

(57) ABSTRACT

The present invention relates to a process for preparing compounds of the formula (I)

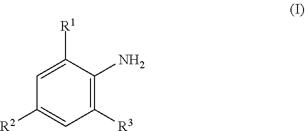

starting from compounds of the formula (II)

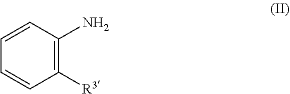

wherein R¹, R², R³ and R^{3′} are defined in according to the invention.

The present invention relates to a process for preparing compounds of the formula (I)

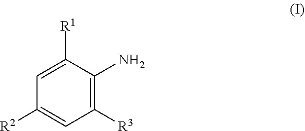

proceeding from compounds of the formula (II)

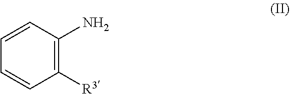

in which R¹, R², R³ and R^{3′} have the meanings described hereinafter.

One possible process for preparing compounds of formula (I) or precursors thereof is described for example in EP1380568 and WO2016/174052. The preparation is effected by perfluoroalkylation in the para position of anilines already substituted in the ortho and meta position. The processes described have the disadvantage that the products are obtained in varying yields that are only moderate in some cases, depending on the substitution, or can be obtained in good yields exclusively by means of very high-waste Fenton oxidation. Moreover, the compounds of the formula (I) have to be prepared in multistage processes. Further possible processes for preparing compounds of the formula (I) are likewise described in WO2016/174052 and also in US2010/0204504, EP2319830 and EP2325165. In a two-stage process, anilines that have first been perfluoroalkylated in the para position and may optionally also have substitution in the ortho position are prepared and isolated here. These may then be halogenated in a further step in the meta position or in the meta and ortho position to give compounds of the formula (I). A particular disadvantage of the processes described is the need to isolate the perfluoroalkylated intermediates. Firstly, this necessitates a complex two-stage process with higher energy expenditure, time demands and incidence of waste. Moreover, the intermediates, owing to their structure, tend to break down easily via polymerization and hence have only limited stability in concentrated form. All the processes described in the prior art additionally have the disadvantage that they are performed in solvents that are undesirable for processes on an industrial scale, such as dimethylformamide, dichloromethane or chloroform.

Substituted anilines of the formula (I) are of great significance as a building block for synthesis of novel active agrochemical ingredients. The problem addressed by the present invention is therefore that of providing a process for preparing compounds of the general formula (I) which can be used on an industrial scale and inexpensively and avoids the disadvantages described above. It is also desirable to obtain the compounds of the formula (I) with high yield and high purity, such that the target compounds preferably do not have to be subjected to any further potentially complex purification.

This problem was solved in accordance with the invention by a process for preparing compounds of the formula (I)

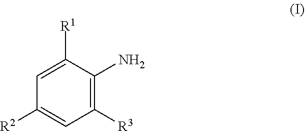

in which
R¹ is chlorine or bromine,
R² is C₁-C₄-haloalkyl and
R³ is cyano, halogen, optionally halogen- or CN-substituted C₁-C₄-alkyl or optionally halogen-substituted C₁-C₄-alkoxy,
proceeding from compounds of the formula (II)

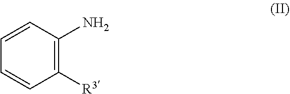

in which R^{3′} is hydrogen, cyano, halogen, optionally halogen- or CN-substituted C₁-C₄-alkyl or optionally halogen-substituted C₁-C₄-alkoxy,
comprising the following steps (1) and (2):
(1) reacting compounds of the formula (II) with compounds of the formula R²—Y where Y is iodine or bromine to give compounds of the formula (III)

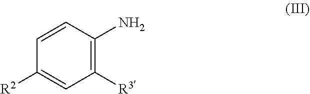

where R² and R^{3′} have the definitions given above and
(2) chlorinating or brominating compounds of the formula (III) with a chlorinating or brominating agent to give compounds of the formula (I),
characterized in that compounds of the formula (III) are not isolated from the reaction mixture from step (1) prior to step (2).

The process according to the invention has the advantage over the process described above that the desired compounds of the formula (I) are obtained in high yields and purities and, at the same time, waste streams and process steps are reduced and the overall process can thus be conducted in a simpler and more efficient and hence less expensive manner Moreover, the process according to the invention enables the complete avoidance of solvents that are undesirable in processes on industrial scale in all steps.

The preferred embodiments described below refer, if appropriate, to all formulae described herein.

In the context of this invention, the term halogen preferably denotes chlorine, fluorine, bromine or iodine, more preferably chlorine, fluorine or bromine.

In a preferred embodiment of the invention,
R² is fluorine-substituted C₁-C₄-alkyl.

More preferably,
R² is perfluoro-C₁-C₃-alkyl (CF₃, C₂F₅ or C₃F₇ (n- or isopropyl)).

Most preferably,
R² is heptafluoroisopropyl.

In a further preferred embodiment,
R³ is a substituent selected from Cl, Br, F, C₁-C₃-alkyl, halogen-substituted C₁-C₃-alkyl, C₁-C₃-alkoxy or halogen-substituted C₁-C₃-alkoxy.

In a particularly preferred embodiment,
R³ is Cl, Br, C₁-C₃-alkyl or fluorine-substituted C₁-C₃-alkyl, C₁-C₃-alkoxy or fluorine-substituted C₁-C₃-alkoxy.

Most preferably,
R³ is Cl, trifluoromethyl, trifluoromethoxy or difluoromethoxy.

In a particularly advantageous configuration of the invention, R¹ and R³ are both chlorine or bromine, especially preferably chlorine.

In a further particularly advantageous configuration of the invention,

R¹ is chlorine or bromine,

R² is perfluoro-C₁-C₃-alkyl and

R³ is halogen, C₁-C₃-alkyl or fluorine-substituted C₁-C₃-alkyl, C₁-C₃-alkoxy or fluorine-substituted C₁-C₃-alkoxy.

In a very particularly advantageous configuration of the invention,
R¹ is chlorine or bromine,

R² is heptafluoroisopropyl and

R³ is Cl, trifluoromethyl, trifluoromethoxy or difluoromethoxy.

In a further preferred embodiment,

R^{3′} is a substituent selected from hydrogen, Cl, Br, F, C₁-C₃-alkyl, halogen-substituted C₁-C₃-alkyl, C₁-C₃-alkoxy or halogen-substituted C₁-C₃-alkoxy.

In a particularly preferred embodiment,

R³ is hydrogen, Cl, Br, C₁-C₃-alkyl or fluorine-substituted C₁-C₃-alkyl, C₁-C₃-alkoxy or fluorine-substituted C₁-C₃-alkoxy.

Most preferably,

R^{3′} is hydrogen, Cl, trifluoromethyl, trifluoromethoxy or difluoromethoxy.

The anilines of the formula (II) used as starting materials are commercially available.

Preference is given here to the following anilines of the formula (II):
aniline,
2-methylaniline,
2-chloroaniline,
2-trifluoromethylaniline,
2-trifluoromethoxyaniline and
2-difluoromethoxyaniline

Particular preference is given here to the following compounds:
aniline,
2-chloroaniline,
2-trifluoromethylaniline,
2-trifluoromethoxyaniline and
2-difluoromethoxyaniline

These compounds preferably give rise to the following compounds of the formula (I):
2,6-dichloro-4-(1,1,1,2,3,3,3-heptafluoropropan-2-yl)aniline,
2-chloro-6-methyl-4-(1,1,1,2,3,3,3-heptafluoropropan-2-yl)aniline,
2-bromo-6-methyl-4-(1,1,1,2,3,3,3-heptafluoropropan-2-yl)aniline,
2-chloro-4-(1,1,1,2,3,3,3-heptafluoropropan-2-yl)-6-(trifluoromethyl)aniline,
2-chloro-4-(1,1,1,2,3,3,3-heptafluoropropan-2-yl)-6-(trifluoromethoxy)aniline,
2-chloro-6-(difluoromethoxy)-4-(1,1,1,2,3,3,3-heptafluoropropan-2-yl)aniline,
2-bromo-4-(1,1,1,2,3,3,3-heptafluoropropan-2-yl)-6-(trifluoromethyl)aniline and
2-bromo-4-(1,1,1,2,3,3,3-heptafluoropropan-2-yl)-6-(trifluoromethoxy)aniline

Particular preference is given to
2,6-dichloro-4-(1,1,1,2,3,3,3-heptafluoropropan-2-yl)aniline,
2-chloro-4-(1,1,1,2,3,3,3-heptafluoropropan-2-yl)-6-(trifluoromethyl)aniline,
2-chloro-4-(1,1,1,2,3,3,3-heptafluoropropan-2-yl)-6-(trifluoromethoxy)aniline,
2-chloro-6-(difluoromethoxy)-4-(1,1,1,2,3,3,3-heptafluoropropan-2-yl)aniline and
2-bromo-4-(1,1,1,2,3,3,3-heptafluoropropan-2-yl)-6-(trifluoromethoxy)aniline

In the context of the present invention, unless defined differently elsewhere, the term “alkyl”, according to the invention, either on its own or else in combination with further terms, for example haloalkyl, is understood to mean a radical of a saturated, aliphatic hydrocarbon group which has 1 to 12, preferably 1 to 6 and more preferably 1 to 4 carbon atoms and may be branched or unbranched. Examples of C₁-C₁₂-alkyl radicals are methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1-ethylpropyl, 1,2-dimethylpropyl, hexyl, n-heptyl, n-octyl, n-nonyl, n-decyl, n-undecyl and n-dodecyl.

The term “alkoxy”, either on its own or else in combination with further terms, for example haloalkoxy, is understood in the present case to mean an O-alkyl radical where the term “alkyl” is as defined above.

According to the invention, unless defined differently elsewhere, the term “aryl” is understood to mean an aromatic radical having 6 to 14 carbon atoms, preferably phenyl, naphthyl, anthryl or phenanthrenyl, more preferably phenyl.

Halogen-substituted radicals, for example haloalkyl, are mono- or polyhalogenated up to the maximum number of possible substituents. In the case of polyhalogenation, the halogen atoms may be identical or different. Unless stated otherwise, optionally substituted radicals may be mono- or polysubstituted, where the substituents in the case of polysubstitutions may be the same or different.

The ranges specified above generally or in preferred ranges apply correspondingly to the overall process. These definitions can be combined with one another as desired, i.e. including combinations between the respective preferred ranges.

Preference is given in accordance with the invention to using processes in which there is a combination of the meanings and ranges specified above as being preferred.

Particular preference is given in accordance with the invention to using processes in which there is a combination of the meanings and ranges specified above as being particularly preferred.

Very particular preference is given in accordance with the invention to using processes in which there is a combination of the meanings and ranges specified above as being very particularly preferred.

Especially used in accordance with the invention are processes in which there is a combination of the meanings and ranges specified above with the term “especially”.

Specifically used in accordance with the invention are processes in which there is a combination of the meanings and ranges specified above with the term “specifically”.

### PROCESS DESCRIPTION

Step (1):

According to the invention, the compounds of the formula (II) are reacted with compounds of the formula R²—Y where Y is iodine or bromine to give compounds of the formula (III)

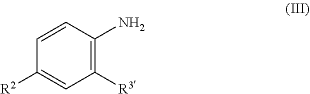

where R² and R^{3′} have the definitions given above.

According to the invention, preference is given here to using between 0.9 and 2.0 equivalents, more preferably between 1.0 and 1.8 equivalents, most preferably between 1.0 and 1.5 equivalents, based on the total molar amount of the compounds of the formula (II) used, of the compounds of the formula R²—Y. Although the use of larger excesses is chemically possible, it is not expedient from an economic point of view.

The compounds of the formula R²—Y are used here in pure form, or as a solution in the solvent preferred for the reaction in concentrations of 40-95% by weight, more preferably in pure form or as a solution in any preferred organic solvent in concentrations of 60-90% by weight and most preferably in pure form or as a solution in a preferred solvent in concentrations of 60-85% by weight.

In a preferred embodiment of the invention,

Y is iodine.

Preferred compounds of the formula R²—Y are especially pentafluoroiodoethane, heptafluoro-1-iodopropane, heptafluoro-2-iodopropane and heptafluoro-2-bromopropane, particular preference being given to heptafluoro-2-iodopropane and heptafluoro-2-bromopropane, very particular preference to heptafluoro-2-iodopropane.

The compounds of the formula (III) can be prepared in step (1) from the corresponding anilines, for example in analogy to the methods described in JP 2012/153635 A and CN 106748807 A.

In step (1), preference is given to using a suitable organic solvent. Suitable solvents are, for example: aromatic or aliphatic halohydrocarbons, especially aromatic or aliphatic chlorohydrocarbons, such as tetrachloroethane, dichloropropane, dichloromethane, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene and trichlorobenzene; esters, especially methyl acetate, ethyl acetate, propyl (n- and iso-) acetate or butyl acetate; ethers, especially tetrahydrofuran (THF), 2-methyl-THF, cyclopentyl methyl ether, tert-butyl methyl ether or diethyl ether; optionally substituted aliphatic, cycloaliphatic or aromatic hydrocarbons, especially pentane, hexane, heptane, octane, nonane, cyclohexane, methylcyclohexane, petroleum ether, ligroin, benzene, toluene, anisole, xylene, mesitylene or nitrobenzene; and also nitriles, especially acetonitrile or propionitrile.

Preferred solvents are acetonitrile, methyl acetate, ethyl acetate, isopropyl acetate, tert-butyl methyl ether, cyclopentyl methyl ether, THF and methyl-THF. Very particular preference is given to acetonitrile, tert-butyl methyl ether, ethyl acetate and isopropyl acetate.

The solvents may be used alone or in a combination of two or more.

Step (1) is preferably performed in a biphasic system composed of one of the abovementioned organic solvents according to the invention and water, for example in a ratio of 5:1 to 1:5 (organic solvent:water), more preferably in a ratio of 5:1 to 1:2, most preferably in a ratio of 2:1 to 1:2.

Preference is given to performing step (1) in the presence of a phase transfer catalyst preferably selected from quaternary ammonium salts (especially tetra-n-butylammonium hydrogensulfate, chloride or bromide) and tetraalkylphosphonium salts (especially tri-n-butyl(tetradecyl)butylphosphonium chloride or trihexyltetradecylphosphonium chloride). The phase transfer catalyst is more preferably selected from tetra-n-butylammonium hydrogensulfate and tri-n-hexyltetradecylphosphonium chloride.

According to the invention, the phase transfer catalyst is preferably used in a proportion between 0.005 and 0.06 equivalent, more preferably in a proportion between 0.01 and 0.05 equivalent, based on the total molar amount of compound (II) used. The catalyst is preferably used here in pure form.

Step (1) is preferably performed in the presence of a reducing agent, for example sodium dithionite or potassium dithionite, more preferably sodium dithionite. According to the invention, preference is given here to using between 0.9 and 2.0 equivalents, more preferably between 1.0 and 1.8 equivalents, most preferably between 1.0 and 1.5 equivalents, based on the total molar amount of compound (II) used. The reducing agent is preferably used here in pure form.

Step (1) is preferably performed at an ambient temperature in the range from −10° C. to 80° C., more preferably in the range from 0° C. to 60° C. and most preferably in the range from 5° C. to 40° C.

Step (1) is preferably performed in the region of standard pressure (1013 hPa), for example in the range from 300 hPa to 5000 hPa or from 500 hPa to 2000 hPa, preferably as in the region of 1013 hPa±200 hPa.

The reaction time for the perfluoroalkylation in step (1) is preferably in the range from 3 to 48 hours, more preferably between 3 and 24 hours, most preferably between 6 and 24 hours.

The compounds R²—Y are preferably added by continuous metered addition over a period of 2 to 10 hours, more preferably between 3 and 6 hours.

Step (1) is preferably performed with pH monitoring. The pH of the reaction solution is preferably kept here within a pH range between 3 and 7, more preferably within a pH range between 4 and 7. The pH is preferably monitored both during the addition of the compounds R²—Y and during the subsequent reaction over the entire reaction time and by addition of a suitable base commonly known to the person skilled in the art, for example as a pure substance or aqueous solutions of alkali metal/alkaline earth metal carbonates, alkali metal/alkaline earth metal hydrogencarbonates or alkali metal/alkaline earth metal hydroxides. In some cases, it may be advantageous to adjust the pH of the reaction mixture prior to commencement of the metered addition of the compounds R²—Y to a preferred pH, especially a pH of 4 to 5, by addition of a suitable acid commonly known to the person skilled in the art, for example carboxylic acids, for example acetic acid or propionic acid, mineral acids, for example hydrochloric acid or sulfuric acid, or sulfonic acids, for example methanesulfonic acid.

Step (2), Chlorination/Bromination:

According to the invention, the compounds of the formula (III) are reacted with a chlorinating or brominating agent to give compounds of the formula (I).

In the context of this description of the invention, the term halogenating agents is used to represent chlorinating agents or brominating agents.

In the section of the description which follows, relating to step (2), the term halogen represents chlorine or bromine.

Suitable halogenating agents are the halogenating agents that are common knowledge to the person skilled in the art, for example chlorine, bromine, an inorganic chlorine- or bromine-containing salt, or an organic chlorine- or bromine-containing molecule, in which the bond of an organic radical to the halogen atom is polarized, such that the chlorine or bromine atom is the carrier of a partially positive charge, for example N-halosuccinimides, 1,3-dihalo-5,5-dimethylhydantoins or halocyanuric acids (organic halogenating compounds).

Suitable halogenating agents here are chlorine, bromine or organic halogenating agents that are more preferably selected from N-chlorosuccinimide (NCS), N-bromosuccinimide (NBS), 1,3-dichloro-5,5-dimethylhydantoin (DCDMH), 1,3-dibromo-5,5-dimethylhydantoin (DBDMH), 1,3,5-trichloro-1,3,5-triazine-2,4,6-trione (TCCA), 1,3,5-tribromo-1,3,5-triazine-2,4,6-trione or 1,3-dibromo-1,3,5-triazine-2,4,6-trione. Most preferably, the halogenating compounds are selected from chlorine, bromine, 1,3-dichloro-5,5-dimethylhydantoin (DCDMH), 1,3-dibromo-5,5-dimethylhydantoin (DBDMH), 1,3,5-trichloro-1,3,5-triazine-2,4,6-trione, 1,3,5-tribromo-1,3,5-triazine-2,4,6-trione or 1,3-dibromo-1,3,5-triazine-2,4,6-trione, especially preferably chlorine, bromine, 1,3-dibromo-5,5-dimethylhydantoin (DBDMH) or 1,3,5-trichloro-1,3,5-triazine-2,4,6-trione (TCCA).

The halogenating agents may be used alone or in a combination of two or more, provided that the compounds used bear the same halogen.

According to the invention, the halogenating agent may be used in a proportion of between 1.0 and 3.0 equivalents (monohalo compounds) or between 0.5 and 1.5 equivalents (dihalo compounds) or 0.3 and 1.0 equivalent (trihalo compounds), and preferably between 1.0 and 2.5 equivalents (monohalo compounds) or between 0.5 and 0.8 equivalent (dihalo compounds) or between 0.33 and 0.75 equivalent (trihalo compounds), based on the total molar amount of compound (III) used. If appropriate, it is possible to neutralize an excess of the halogenating agent after full conversion, detected by means of HPLC^{a}, by the addition of a reducing agent commonly known to the person skilled in the art, for example alkali metal/alkaline earth metal sulfites, alkali metal/alkaline earth metal dithionites or alkali metal/alkaline earth metal thiosulfates. The reducing agents may preferably be used here as a pure substance or as an aqueous solution, for example as a saturated aqueous solution. ^{a)} HPLC (High Performance Liquid Chromatography) on a reverse-phase column (C18), Agilent 1100 LC system; Phenomenex Prodigy 100×4 mm ODS3; eluent A: acetonitrile (0.25 ml/l); eluent B: water (0.25 ml TFA/1); linear gradient from 5% acetonitrile to 95% acetonitrile in 7.00 min, then 95% acetonitrile for a further 1.00 min; oven temperature 40° C.; flow rate: 2.0 ml/min.

According to the invention, the halogenating agent may be in pure form as a solid or as a suspension or solution in a suitable organic solvent which is inert under the reaction conditions, especially in the solvent selected for the reaction, preferably at a concentration of 40-90% by weight, more preferably at a concentration of 60-95% by weight. Suitable organic solvents are especially the preferred solvents mentioned below for step (2).

No particular catalysts are needed for step (2). Under some circumstances, it may be advantageous to utilize acids in catalytic amounts for activation, but this is not absolutely necessary in the reactions claimed here. More particularly, this is advantageous in the case of use of organic chlorinating agents, for example N-chlorosuccinimide (NCS) and 1,3-dichloro-5,5-dimethylhydantoin (DCDMH).

Suitable acids may preferably be selected from the mineral acids familiar to the person skilled in the art, for example sulfuric acid, hydrochloric acid and hydrofluoric acid, sulfonic acids, for example methanesulfonic acid, trifluoromethanesulfonic acid and 4-toluenesulfonic acid, carboxylic acids, for example trifluoroacetic acid and trichloroacetic acid, and Lewis acids, for example iron(III) trifluoromethanesulfonate and scandium(III) trifluoromethanesulfonate.

The reaction is preferably conducted within a temperature range from −78 to 200° C., more preferably at temperatures between −20 and 100° C. and most preferably between 0° C. and 50° C.

The reaction may be performed at elevated or else reduced pressure. However, it is preferably conducted at standard pressure, for example in the region of 1013 hPa±300 hPa, or in the region of 1013 hPa±100 hPa, or in the region of 1013 hPa±50 hPa.

Step (2) is preferably conducted in a suitable organic solvent. Useful diluents or solvents for conducting step (2) in principle include organic solvents that are inert under the specific reaction conditions.

Examples include: aromatic or aliphatic halohydrocarbons, especially aromatic or aliphatic chlorohydrocarbons, such as tetrachloroethane, dichloropropane, dichloromethane, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene or trichlorobenzene; nitriles, especially acetonitrile, propionitrile, butyronitrile, isobutyronitrile, benzonitrile or m-chlorobenzonitrile; optionally substituted aliphatic, cycloaliphatic or aromatic hydrocarbons, especially pentane, hexane, heptane, octane, nonane, cyclohexane, methylcyclohexane, petroleum ether, ligroin or nitrobenzene; esters, especially methyl acetate, ethyl acetate, isopropyl acetate, butyl acetate, isobutyl acetate, dimethyl carbonate, dibutyl carbonate or ethylene carbonate; amides, especially N,N-dimethylformamide (DMF), N,N-dipropylformamide, N,N-dibutylformamide (DBF), N,N-dimethylacetamide (DMAC) or N-methylpyrrolidine (NMP); aliphatic or cycloaliphatic ethers, especially 1,2-dimethoxyethane (DME), diglyme, tetrahydrofuran (THF), 2-methyl-THF, 1,4-dioxane, tert-butyl methyl ether or cyclopentyl methyl ether and carboxylic acids, especially acetic acid, n-propanoic acid or n-butanoic acid.

Preferred diluents or solvents are aromatic or aliphatic halogenated hydrocarbons, especially chlorobenzene, dichlorobenzene, dichloromethane, chloroform, 1,2-dichloroethane or carbon tetrachloride; esters, especially ethyl acetate, isopropyl acetate and butyl acetate; amides, especially DMF, DMAC and NMP; ethers, especially tetrahydrofuran (THF), 2-methyl-THF, tert-butyl methyl ether or cyclopentyl methyl ether; nitriles, especially acetonitrile or propionitrile, or carboxylic acids, especially acetic acid or n-propanoic acid.

In a very particularly preferred embodiment, the solvent is selected from ethyl acetate, isopropyl acetate, tert-butyl methyl ether, cyclopentyl methyl ether, THF, 2-methyl-THF and acetonitrile. Very particular preference is given to acetonitrile, tert-butyl methyl ether, ethyl acetate and isopropyl acetate.

The solvents may be used alone or in a combination of two or more.

The duration of the halogenation of the compounds of the formula (III) is preferably in the range from 0.5 h to 10 h, more preferably in the range from 0.25 h to 5 h. A longer reaction time is possible but is not expedient from an economic point of view.

The halogenating agent may be added to the other reactants in one portion or by metered addition over a prolonged period. Under some circumstances, it may also be advantageous to meter a solution of the compound (III) in one of the solvents mentioned for step (2) into a solution or suspension of the halogenating agent in one of the solvents preferred for step (2). The duration of the metered addition here may be within a preferred range from 0.5 to 6 hours, more preferably from 1 to 4 hours. Longer metering times are also possible from a technical point of view but are not expedient from an economic point of view.

The (metered) addition is preferably effected within a temperature range from −78 to 200° C., more preferably at temperatures from −20 to 100° C. and most preferably between 0° C. and 50° C. In an advantageous configuration, the temperature at which metered addition is effected corresponds to the reaction temperature.

In a particularly advantageous configuration of the invention, the same organic solvent is used in step (1) and step (2).

In the context of this configuration of the invention, the solvent in both steps is preferably selected from the group of the esters, ethers or the nitriles; the solvent is more preferably selected from ethyl acetate, isopropyl acetate, tert-butyl methyl ether, cyclopentyl methyl ether, THF, methyl-THF and acetonitrile. Very particular preference is given to acetonitrile, tert-butyl methyl ether, ethyl acetate and isopropyl acetate.

The solvents mentioned may be used alone or in a combination of two or more.

It is a feature of the process according to the invention that compounds of the formula (III) are not isolated from the reaction mixture from step (1) prior to step (2).

The term “isolating” in the context of the present invention means complete separation of the compounds of the formula (III) from the reaction mixture, i.e., for example, from all solvents and salts, by separation methods that are common knowledge to the person skilled in the art. In addition, “isolating” in the context of the present invention means that all the organic solvent from step (1) is never removed after step (1) and before step (2).

Preferably, the compounds of the formula (III) from step (1) are used in step (2) directly as a solution in the organic solvent from step (1).

In the process according to the invention, during the reaction sequence, reaction volumes may be added in the form of solids, liquids or suspensions, for example in the form of solid, dissolved or suspended halogenating agents, or solvents (the same solvent as in the first step or a further solvent). More particularly, addition of acids or bases and the partial or complete removal of aqueous constituents of the reaction mixture between reaction steps (1) and (2) is possible.

According to the invention, it is further preferable that less than 30% by volume, more preferably less than 20% by volume and most preferably less than 10% by volume of the organic solvent from step (1) is removed before the start of step (2), based on the volume of organic solvent used.

It is especially advantageous when no organic solvent is actively removed after step (1). Active removal of the organic solvent is generally understood to mean the removing of the organic solvent by means of distillation, optionally by thermal treatment of the reaction mixture, under standard or reduced pressure.

In a further preferred configuration of the invention, step (1) and step (2) are effected in the same reaction vessel. In this case, the person skilled in the art will choose a reaction vessel from the outset that can accommodate all volumes for reaction (1) and (2).

In other words, it is preferable for the reaction sequence to be a telescoped reaction in one or more vessels, preferably one vessel.

The process according to the invention preferably consists of steps (1) and (2).

Optionally, step (1) and/or step (2) can also be performed repeatedly, for example two or three times, in the same reaction vessel without further workup. The reaction mixture from step (1) can, for example, after complete conversion according to HPLC^{a}, be admixed again with the compound of the formula (II) and a reducing agent according to the invention and converted to a compound of the formula (III) by metered addition of a compound R²—Y with pH monitoring. This operation can be repeated again, or the reaction mixture can be treated further in accordance with the invention. The reaction mixture from step (2) can analogously, after complete conversion according to HPLC^{a}, be admixed again with compound of the formula (III) and then converted further to compounds of the formula (I) by addition of a halogenating agent according to the invention.

The compounds (I) can be worked up and isolated after complete reaction, for example, by the removal of solvent, washing with water and extraction with a suitable organic solvent and separation of the organic phase, and removal of the solvent under reduced pressure. The residue can also be subjected to a vacuum distillation at 0.05-1 bar with a concentric tube fractionation column and a crystallization in a solvent commonly known to the person skilled in the art.

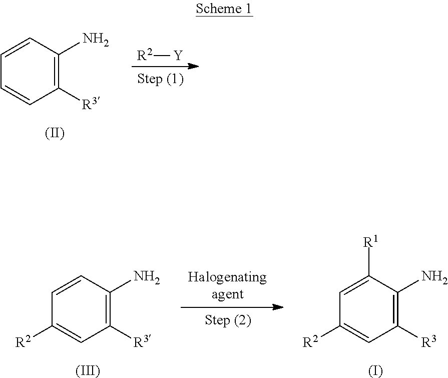

Scheme 1 gives a schematic overall representation of the process according to the invention with both steps. Reaction conditions and reactants are selected here in accordance with the above-described inventive and preferred configurations. All variables in the formulae (I), (II), (III) and R²—Y are defined as described above.

A preferred embodiment of the process according to the invention is as follows:

The compounds of the formula (II) are initially charged in a mixture of an organic solvent and water and, after addition of a phase transfer catalyst according to the invention, e.g. tetra-n-butylammonium hydrogensulfate or tri-n-hexyl(tetradecyl)phosphonium chloride, and a reducing agent according to the invention, e.g. sodium dithionite, a perfluoroalkylating agent according to the invention, e.g. heptafluoro-2-iodopropane, is added at preferably −10° C. to 80° C., more preferably 0° C. to 60° C., over the course of 2 h to 10 h, optionally after the pH has been adjusted to 4 to 5 prior to commencement of the metered addition by a suitable acid, for example acetic acid. The pH of the reaction mixture is kept here within a range from 3 to 7 over the entire reaction time, preferably by addition of a suitable base, in solid form or as an aqueous solution, for example 40% by weight aqueous potassium carbonate solution. After preferably 3 h to 48 h, the aqueous phase is removed, the organic phase is optionally washed with water or aqueous hydrochloric acid, e.g. 5% by weight or 25% by weight, and the organic phase containing compounds of the formula (III) is admixed with a halogenating agent, for example in solid form or as a solution in an organic solvent according to the invention, preferably at −20° C. to 100° C., more preferably at 0° C. to 50° C., over preferably 0.5 h to 6 h. On completion of conversion (HPLC^{a}), any excess halogenating agent present is neutralized by the addition of a reducing agent, for example as a pure substance or aqueous solution, and the compounds of the formula (I) are isolated. (Step (1) and (2)).

In a further advantageous embodiment, the compounds of the formula (II) are initially charged in a mixture of an organic solvent and water and, after addition of a phase transfer catalyst according to the invention, e.g. tetra-n-butylammonium hydrogensulfate or tri-n-hexyl(tetradecyl)phosphonium chloride, and a reducing agent according to the invention, e.g. sodium dithionite, a perfluoroalkylating agent according to the invention, e.g. heptafluoro-2-iodopropane, is added at preferably −10° C. to 80° C., more preferably 0° C. to 60° C., over the course of 2 h to 10 h, optionally after the pH has been adjusted to 4 to 5 prior to commencement of the metered addition by a suitable acid, for example acetic acid. The pH of the reaction mixture is kept here within a range from 3 to 7 over the entire reaction time, preferably by addition of a suitable base, in solid form or as an aqueous solution, for example 40% by weight aqueous potassium carbonate solution. After preferably 3 h to 48 h, after addition of a further portion of the compound of the formula (II) and of a reducing agent according to the invention, e.g. sodium dithionite, a perfluoroalkylating agent according to the invention, e.g. heptafluoro-2-iodopropane, is added at preferably −10° C. to 80° C., more preferably 0° C. to 60° C., over 2 h to 10 h. The pH of the reaction mixture is kept here within a range from 3 to 7 over the entire reaction time, preferably by addition of a suitable base, in solid form or as an aqueous solution, for example aqueous potassium carbonate solution. After preferably 3 h to 48 h, the process can optionally be repeated again or the aqueous phase can be removed, the organic phase can optionally be washed with water or aqueous hydrochloric acid, e.g. 5% by weight or 25% by weight, and the organic phase containing compounds of the formula (III) can be admixed with a halogenating agent, for example in solid form or as a solution in an organic solvent according to the invention, preferably at −20° C. to 100° C., more preferably at 0° C. to 50° C., over preferably 0.5 h to 6 h. On completion of conversion (HPLC^{a}), any excess halogenating agent present is neutralized by the addition of a reducing agent, for example as a pure substance or aqueous solution, and the compounds of the formula (I) are isolated. (Step (1) (twice) and (2)).

A particularly preferred embodiment of the process according to the invention is as follows:

The compounds of the formula (II) are initially charged in a mixture of ethyl acetate and water and, after addition of tetra-n-butylammonium hydrogensulfate and sodium dithionite, heptafluoro-2-iodopropane is added at 0° C. to 60° C. over 3 h to 6 h, optionally after the pH has been adjusted to 4 to 5 with acetic acid prior to commencement of the metered addition. The pH of the reaction mixture is kept here within a range from 4 to 7 over the entire metering and reaction time by addition of a 40% by weight aqueous potassium carbonate solution. After preferably 3 h to 24 h, the aqueous phase is removed, the organic phase is optionally washed with water or aqueous hydrochloric acid, for example 5% by weight or 25% by weight, and the organic phase containing compounds of the formula (III) is admixed with chlorine or 1,3,5-trichloro-1,3,5-triazine-2,4,6-dione (TCCA) (chlorination) or bromine or 1,3-dibromo-5,5-dimethylhydantoin (DBDMH) (bromination), preferably at 0° C. to 50° C., over the course of 1 h to 4 h. On completion of conversion (HPLC^{a}), any excess halogenating agent present is neutralized by the addition of sodium sulfite, as a pure substance or as an aqueous solution, and the compounds of the formula (I) are isolated. (Step (1) and (2)).

### EXAMPLES

The examples which follow elucidate the process according to the invention in detail without restricting the invention thereto.

Methods:

The NMR data of the examples are listed in conventional form (δ values, multiplet splitting, number of hydrogen atoms).

The solvent and the frequency in which the NMR spectrum was recorded are stated in each case.

Step 1: Preparation of the Compounds of the Formula (III)

### 4-[1,2,2,2-Tetrafluoro-1-(trifluoromethyl)ethyl]aniline (III-1a)

To an initial charge of 60.0 g (0.64 mol, 1.0 eq) of aniline in 450 ml each of water and ethyl acetate were successively added 4.5 g (13.0 mmol, 0.02 eq) of tetra-n-butylammonium hydrogensulfate and 144.0 g (0.70 mol, 1.1 eq, 85% by weight) of sodium dithionite. 214.0 g (0.70 mol, 1.1 eq) of heptafluoro-2-iodopropane was metered in at room temperature over the course of 3 h and the pH was kept at 6.0-7.0 during the metered addition by adding 40% by weight aqueous K₂CO₃. On completion of addition, stirring was continued at the same pH at about 21° C. for another 3 h, then the phases were separated, and the organic phase was washed with a solution of 40 ml each of 20% by weight NaCl and 2.5% by weight HCl. By means of HPLC^{a}), a conversion of 98% to the desired product was detected. The organic phase was then used without further treatment in step (2).

An analytical sample of the pure compound was obtained after isolation by distillative removal of the solvent.

¹H-NMR (CDCl₃, 400 MHz) δ (ppm)=7.35 (d, J=8.9 Hz, 2H), 6.72 (d, J=7.7 Hz, 2H), 3.91 (br s, 2H).

### 4-[1,2,2,2-Tetrafluoro-1-(trifluoromethyl)ethyl]aniline (III-1 b)

To an initial charge of 7.5 g (79.8 mmol, 1.0 eq) of aniline in 60 ml each of water and ethyl acetate were successively added 0.1 g (0.4 mmol, 0,005 eq) of tetra-n-butylammonium hydrogensulfate and 17.9 g (87.8 mol, 1.1 eq, 85% by weight) of sodium dithionite. 26.8 g (87.8 mmol, 1.1 eq) of heptafluoro-2-iodopropane, diluted with 8 ml of ethyl acetate, was metered in at 20-22° C. over the course of 4 h and the pH was kept at 6.0-7.0 during the metered addition by adding 40% by weight aqueous K₂CO₃. On completion of addition, the mixture was stirred at about 20-22° C. at the same pH for a further 1.5 h. By means of HPLC^{a}), a conversion of 98% to the desired product was detected. The phases were separated, and the organic phase was washed with 75 ml of 10% by weight HCl. The organic phase was then used without further treatment in step (2).

An analytical sample of the pure compound was obtained after isolation by distillative removal of the solvent.

¹H-NMR (CDCl₃, 400 MHz) δ (ppm)=7.35 (d, J=8.9 Hz, 2H), 6.72 (d, J=7.7 Hz, 2H), 3.91 (br s, 2H).

### 4-[1,2,2,2-Tetrafluoro-1-(trifluoromethyl)ethyl]aniline (III-1c)

To an initial charge of 7.5 g (79.8 mmol, 1.0 eq) of aniline in 60 ml each of water and ethyl acetate were successively added 1.4 g (1.6 mmol, 0.02 eq) of tri-n-butyl(tetradecyl)phosphonium chloride and 17.9 g (87.8 mol, 1.1 eq, 85% by weight) of sodium dithionite. 26.8 g (87.8 mmol, 1.1 eq) of heptafluoro-2-iodopropane, diluted with 8 ml of ethyl acetate, was metered in at 20-22° C. over the course of 3 h and the pH was kept at 6.0-7.0 during the metered addition by adding 40% by weight aqueous K₂CO₃. On completion of addition, the mixture was stirred at about 20-22° C. at the same pH for a further 4 h. By means of HPLC^{a}), a conversion of 96% to the desired product was detected. The phases were separated, and the organic phase was washed with 75 ml of 10% by weight HCl. The organic phase was then used without further treatment in step (2).

An analytical sample of the pure compound was obtained after isolation by distillative removal of the solvent.

¹H-NMR (CDCl₃, 400 MHz) δ (ppm)=7.35 (d, J=8.9 Hz, 2H), 6.72 (d, J=7.7 Hz, 2H), 3.91 (br s, 2H).

### 4-[1,2,2,2-Tetrafluoro-1-(trifluoromethyl)ethyl]aniline (III-1d)

To an initial charge of 15.0 g (150.0 mmol, 1.0 eq) of aniline in 120 ml each of water and isopropyl acetate were successively added 3.3 g (9.7 mmol, 0.06 eq) of tetra-n-butylammonium hydrogensulfate and 35.9 g (170.0 mol, 1.1 eq, 85% by weight) of sodium dithionite. 53.51 g (170.0 mmol, 1.1 eq) of heptafluoro-2-iodopropane was metered in at 20-22° C. over the course of 3 h and the pH was kept at 6.0-7.0 during the metered addition by adding 40% by weight aqueous K₂CO₃. On completion of addition, the mixture was stirred at about 20-22° C. at the same pH for a further 5 h. By means of HPLC^{a}), a conversion of 94% to the desired product was detected. The phases were separated and the organic phase was then used without further treatment in step (2).

An analytical sample of the pure compound was obtained after isolation by distillative removal of the solvent.

¹H-NMR (CDCl₃, 400 MHz) δ (ppm)=7.35 (d, J=8.9 Hz, 2H), 6.72 (d, J=7.7 Hz, 2H), 3.91 (br s, 2H).

### 4-[1,2,2,2-Tetrafluoro-1-(trifluoromethyl)ethyl]aniline (III-1e)

To an initial charge of 30.0 g (0.31 mol, 1.0 eq) of aniline in 240 ml each of water and ethyl acetate were successively added 2.2 g (6.2 mmol, 0.02 eq) of tetra-n-butylammonium hydrogensulfate and 71.9 g (0.35 mol, 1.1 eq, 85% by weight) of sodium dithionite. 90.0 g (0.35 mol, 1.1 eq) of heptafluoro-2-bromopropane was metered in by means of a gas introduction tube at −5° C. over the course of 3 h and the pH was kept at 6.0-7.0 during the metered addition by adding 40% by weight aqueous K₂CO₃. On completion of addition, the mixture was stirred at about −5° C. at the same pH for another 3 h and then warmed to 20° C. overnight. By means of HPLC^{a}), a conversion of 96% to the desired product was detected. The phases were separated, and the organic phase was washed with a solution of 40 ml each of 20% by weight NaCl and 2.5% by weight HCl. The organic phase was then used without further treatment in step (2).

An analytical sample of the pure compound was obtained after isolation by distillative removal of the solvent.

¹H-NMR (CDCl₃, 400 MHz) δ (ppm)=7.35 (d, J=8.9 Hz, 2H), 6.72 (d, J=7.7 Hz, 2H), 3.91 (br s, 2H).

### 2-Chloro-4-[1,2,2,2-tetrafluoro-1-(trifluoromethyl)ethyl]aniline (III-2a)

To an initial charge of 10.0 g (78.3 mmol, 1.0 eq) of 2-chloroaniline in 100 ml each of water and ethyl acetate were successively added 0.5 g (1.6 mmol, 0.02 eq) of tetra-n-butylammonium hydrogensulfate and 19.3 g (94.0 mmol, 1.2 eq, 85% by weight) of sodium dithionite. By addition of 1.25 g (20.8 mmol, 0.3 eq) of acetic acid, the pH was adjusted to 5. 26.3 g (86.2 mmol, 1.1 eq) of heptafluoro-2-iodopropane, diluted with 6 ml of ethyl acetate, was metered in at 20-22° C. over the course of 3 h and the pH was kept at 4.0-5.0 during the metered addition by adding 40% by weight aqueous K₂CO₃. On completion of addition, the mixture was stirred at about 20-22° C. at the same pH for a further 4 h. By means of HPLC^{a}), a conversion of 94% to the desired product was detected. The phases were separated, and the organic phase was washed with 75 ml of 10% by weight HCl. The organic phase was then used without further treatment in step (2).

An analytical sample of the pure compound was obtained after isolation by distillative removal of the solvent.

¹H-NMR (CDCl₃, 400 MHz) δ (ppm)=7.47 (s, 1H), 7.28 (d, J=8.0 Hz, 1H), 6.81 (d, J=8.0 Hz, 1H), 4.13 (br s, 2H). 2-Chloro-4-[1,2,2,2-tetrafluoro-1-(trifluoromethyl)ethyl]aniline (III-2b)

To an initial charge of 40.0 g (0.31 mol, 1.0 eq) of 2-chloroaniline in 400 ml each of water and ethyl acetate were successively added 2.2 g (6.3 mmol, 0.02 eq) of tetra-n-butylammonium hydrogensulfate and 77.1 g (0.38 mmol, 1.2 eq, 85% by weight) of sodium dithionite. By addition of 4.8 g (79.9 mmol, 0.25 eq) of acetic acid, the pH was adjusted to 5. 114.8 g (0.38 mol, 1.2 eq) of heptafluoro-2-iodopropane, diluted with 26 ml of ethyl acetate, was metered in at 20-22° C. over the course of 3 h and the pH was kept at 4.0-5.0 during the metered addition by adding 40% by weight aqueous K₂CO₃. On completion of addition, the mixture was stirred at about 20-22° C. at the same pH for a further 4 h. By means of HPLC^{a}), a conversion of 99% to the desired product was detected. The phases were separated, and the organic phase was washed with 300 ml of 10% by weight HCl. The organic phase was then used without further treatment in step (2).

An analytical sample of the pure compound was obtained after isolation by distillative removal of the solvent.

¹H-NMR (CDCl₃, 400 MHz) δ (ppm)=7.47 (s, 1H), 7.28 (d, J=8.0 Hz, 1H), 6.81 (d, J=8.0 Hz, 1H), 4.13 (br s, 2H).

### 2-Chloro-4-[1,2,2,2-tetrafluoro-1-(trifluoromethyl)ethyl]aniline (III-2c)

To an initial charge of 10.0 g (78.3 mmol, 1.0 eq) of 2-chloroaniline in 100 ml each of water and tert-butyl methyl ether were successively added 0.5 g (1.6 mmol, 0.02 eq) of tetra-n-butylammonium hydrogensulfate and 19.3 g (94.0 mmol, 1.2 eq, 85% by weight) of sodium dithionite. By addition of 1.0 g (16.6 mmol, 0.2 eq) of acetic acid, the pH was adjusted to 5. 26.3 g (86.2 mmol, 1.1 eq) of heptafluoro-2-iodopropane, diluted with 6 ml of ethyl acetate, was metered in at 20-22° C. over the course of 3 h and the pH was kept at 4.0-5.0 during the metered addition by adding 40% by weight aqueous K₂CO₃. On completion of addition, the mixture was stirred at about 20-22° C. at the same pH for a further 4 h. By means of HPLC^{a}), a conversion of 82% to the desired product was detected. The phases were separated, and the organic phase was washed with 75 ml of 10% by weight HCl. The organic phase was then used without further treatment in step (2).

An analytical sample of the pure compound was obtained after isolation by distillative removal of the solvent.

¹H-NMR (CDCl₃, 400 MHz) δ (ppm)=7.47 (s, 1H), 7.28 (d, J=8.0 Hz, 1H), 6.81 (d, J=8.0 Hz, 1H), 4.13 (br s, 2H).

### 2-Chloro-4-[1,2,2,2-tetrafluoro-1-(trifluoromethyl)ethyl]aniline (III-2d)

To an initial charge of 15.0 g (0.12 mol, 1.0 eq) of 2-chloroaniline in 120 ml of water and 90 ml of ethyl acetate were successively added 0.8 g (2.4 mmol, 0.02 eq) of tetra-n-butylammonium hydrogensulfate and 28.9 g (0.14 mmol, 1.2 eq, 85% by weight) of sodium dithionite. By addition of 1.9 g (31.6 mmol, 0.3 eq) of acetic acid, the pH was adjusted to 5. 40.1 g (0.13 mmol, 1.1 eq) of heptafluoro-2-iodopropane, diluted with 7 ml of ethyl acetate, was metered in at 20-22° C. over the course of 3 h and the pH was kept at 4.0-5.0 during the metered addition by adding 40% by weight aqueous K₂CO₃. On completion of addition, the mixture was stirred at about 20-22° C. at the same pH for a further 4 h. By means of HPLC^{a}), a conversion of 94% to the desired product was detected. The phases were separated, and the organic phase was washed with 75 ml of 10% by weight HCl. The organic phase was then used without further treatment in step (2).

An analytical sample of the pure compound was obtained after isolation by distillative removal of the solvent.

¹H-NMR (CDCl₃, 400 MHz) δ (ppm)=7.47 (s, 1H), 7.28 (d, J=8.0 Hz, 1H), 6.81 (d, J=8.0 Hz, 1H), 4.13 (br s, 2H).

### 4-[1,2,2,2-Tetrafluoro-1-(trifluoromethyl)ethyl]-2-(trifluoromethoxy)aniline (III-3a)

To an initial charge of 40.0 g (0.22 mol, 1.0 eq) of 2-trifluoromethoxyaniline in 400 ml of water and 250 ml of ethyl acetate were successively added 1.55 g (4.4 mmol, 0.02 eq) of tetra-n-butylammonium hydrogensulfate and 68.0 g (0.33 mol, 1.5 eq) of sodium dithionite. 100.2 g (0.33 mol, 1.5 eq) of heptafluoro-2-iodopropane was metered in at room temperature over the course of 2.5 h and the pH was kept at 4.0-5.0 during the metered addition by adding 40% by weight aqueous K₂CO₃. After addition was complete, stirring was carried out for a further 1 h at approximately 21° C., then the phases were separated. The organic phase was diluted with 100 ml of n-heptane, then washed with 250 ml of 20% by weight HCl, 250 ml of saturated NaCl solution and 250 ml of water. The organic phase was then used without further treatment in step (2).

An analytical sample of the pure compound was obtained after isolation by distillative removal of the solvent.

¹H-NMR (DMSO-d₆, 400 MHz) δ (ppm)=7.51 (d, J=9.0 Hz, 1H), 7.44 (s, 1H), 7.43 (d, J=9.0 Hz, 1H), 6.38 (br s, 2H).

### 4-[1,2,2,2-Tetrafluoro-1-(trifluoromethyl)ethyl]-2-(trifluoromethoxy)aniline (III-3b)

To an initial charge of 40.0 g (0.22 mol, 1.0 eq) of 2-trifluoromethoxyaniline in 600 ml of water and 360 ml of ethyl acetate were successively added 4.6 g (13.5 mmol, 0.06 eq) of tetra-n-butylammonium hydrogensulfate and 59.0 g (0.29 mol, 0.4 eq, 85% by weight) of sodium dithionite. 100.2 g (0.34 mol, 1.5 eq) of heptafluoro-2-iodopropane, dissolved in 20 g of ethyl acetate, was metered in at 25° C. over the course of 1.5 h and the pH was kept at 4.0-4.9 during the metered addition by adding 40% by weight aqueous K₂CO₃. On completion of addition, the mixture was stirred at about 21° C. at the same pH for another 2 h. By means of HPLC^{a}), a conversion of >95% to the desired product was detected. Subsequently, another 40.0 g (0.22 mol, 1.0 eq) of 2-trifluoromethoxyaniline and 59.0 g (0.29 mol, 0.4 eq, 85% by weight) of sodium dithionite were added and, thereafter, over the course of 1.5 h at 25° C., 100.2 g (0.34 mol, 1.5 eq) of heptafluoro-2-iodopropane, dissolved in 20 g of ethyl acetate, was metered in and the pH was kept at 4.0-4.9 during the metered addition by adding 40% by weight aqueous K₂CO₃ and again stirred at 21° C. at the same pH for 2 h. By means of HPLC^{a}), a conversion of >97% to the desired product was detected. The operation was repeated once more with 40.0 g (0.22 mol, 1.0 eq) of 2-trifluoromethoxyaniline, 59.0 g (0.29 mol, 0.4 eq, 85% by weight) of sodium dithionite and 100.2 g (0.34 mol, 1.5 eq) of heptafluoro-2-iodopropane, dissolved in 20 g of ethyl acetate, at a pH of 4.0-4.9 over the course of 1.5 h, followed by continued stirring at a pH of 4.0 to 4.9 for 3 h. By means of HPLC^{a}), a conversion of >97% to the desired product was detected. The phases were separated, and the organic phase, after addition of 400 ml of n-heptane, was washed twice each with 300 ml each time of 20% by weight HCl and once with 300 ml of saturated aqueous NaCl solution. The organic phase was then used without further treatment in step (2).

An analytical sample of the pure compound was obtained after isolation by distillative removal of the solvent.

¹H-NMR (DMSO-d₆, 400 MHz) δ (ppm)=7.51 (d, J=9.0 Hz, 1H), 7.44 (s, 1H), 7.43 (d, J=9.0 Hz, 1H), 6.38 (br s, 2H).

The following 4-perfluoroalkylanilines of the general formula (III) were preparable analogously to examples (III-1a) and (III-1b):

### 4-[1,2,2,2-Tetrafluoro-1-(trifluoromethyl)ethyl]-2-(trifluoromethyl)aniline (III-4)

¹H-NMR (DMSO-d₆, 400 MHz) δ (ppm)=7.51 (d, J=9.0 Hz, 1H), 7.43 (br s, 1H), 7.01 (d, J=9.0 Hz, 1H), 6.38 (br s, 2H).

### 2-Ethyl-4-[1,2,2,2-tetrafluoro-1-(trifluoromethyl)ethyl]aniline (III-5)

¹H-NMR (CDCl₃, 400 MHz) δ (ppm)=7.63 (d, J=8.3 Hz, 1H), 7.53 (br s, 1H), 7.43 (d, J=8.3 Hz, 1H), 2.92 (q, J=7.6 Hz, 2H), 1.35 (t, J=7.6 Hz, 3H).

Step (2): Preparation of the Compounds of the Formula (I)

### 2,6-Dichloro-4-[1,2,2,2-tetrafluoro-1-(trifluoromethyl)ethyl]aniline (I-1a)

180.0 g (0.64 mol, 1.0 eq) of 4-[1,2,2,2-tetrafluoro-1-(trifluoromethyl)ethyl]aniline (III-1) as a solution in 450 ml of ethyl acetate from step (1) (Example (III-1a)), was diluted with a further 150 ml of ethyl acetate and, after addition of 100 ml of water, 96.0 g (128.0 mmol, 2.0 eq) of chlorine gas was added at 0-5° C. over the course of 5 h. The phases were subsequently separated and the aqueous phase was extracted successively with a mixture of 100 ml of ethyl acetate and 50 ml of n-heptane and also a mixture of 50 ml of ethyl acetate and 25 ml of n-heptane. The combined organic phases were washed twice with 100 ml each time of 20% by weight NaCl solution and the product, after removal of the solvent, was obtained as a red-brown oil: yield 200.0 g (95% of theory).

¹H-NMR (CDCl₃, 400 MHz) δ (ppm)=7.41 (s, 2H), 4.76 (br s, 2H).

### 2,6-Dichloro-4-[1,2,2,2-tetrafluoro-1-(trifluoromethyl)ethyl]aniline (I-1b)

To 41.5 g (0.15 mol, 1.0 eq) of 4-[1,2,2,2-tetrafluoro-1-(trifluoromethyl)ethyl]aniline (III-1) as a solution in 120 ml of isopropyl acetate from step (1) (Example (III-1d)) was added 27.0 g (0.38 mol, 2.5 eq) of chlorine gas at 0-5° C. over the course of 4 h. Subsequently, 40 ml of ice-water were added gradually, the phases were separated and the aqueous phase was extracted with 40 ml of isopropyl acetate. The combined organic phases were washed twice with 40 ml each time of 20% by weight NaCl solution and the product, after removal of the solvent, was obtained as a red-brown oil: yield 42.5 g (86% of theory).

¹H-NMR (CDCl₃, 400 MHz) δ (ppm)=7.41 (s, 2H), 4.76 (br s, 2H).

### 2,6-Dichloro-4-[1,2,2,2-tetrafluoro-1-(trifluoromethyl)ethyl]aniline (I-1c)

To 20.8 g (79.7 mmol, 1.0 eq) of 4-[1,2,2,2-tetrafluoro-1-(trifluoromethyl)ethyl]aniline (III-1) as a solution in 50 ml of ethyl acetate from step (1) (Example (III-1c)) was added a solution of 13.1 g (55.7 mmol, 0.7 eq) of 1,3,5-trichloro-1,3,5-triazine-2,4,6-trione (TCCA) in 40 ml of ethyl acetate at 0-5° C. over the course of 2 h. The reaction was warmed to 20-25° C. over the course of 2.5 h and stirred at this temperature for 1 h. The resultant solids were filtered off, and the clear solution was admixed with 10 ml of saturated aqueous Na₂SO₃ solution and 30 ml of water. After separation of the phases, washing of the organic phases with 20 ml of water and 20 ml of saturated NaCl solution, and removal of the solvent under reduced pressure, the product was obtained as a light brown-red oil that solidified on cooling: yield 26.1 g (89.9% of theory).

¹H-NMR (CDCl₃, 400 MHz) δ (ppm)=7.41 (s, 2H), 4.76 (br s, 2H).

### 2,6-Dichloro-4-[1,2,2,2-tetrafluoro-1-(trifluoromethyl)ethyl]aniline (I-1d)

To 46.3 g (0.16 mol, 1.0 eq) of 2-chloro-4-[1,2,2,2-tetrafluoro-1-(trifluoromethyl)ethyl]aniline (III-2) as a solution in 100 ml of ethyl acetate from step (1) (Example (III-2a)) was added a solution of 12.9 g (54.8 mmol, 0.35 eq) of 1,3,5-trichloro-1,3,5-triazine-2,4,6-trione (TCCA) in 50 ml of ethyl acetate at 0-5° C. over the course of 2 h. The reaction was warmed to 20-25° C. over the course of 2.5 h and stirred at this temperature for 1 h. The resultant solids were filtered off, and the clear solution was admixed with 40 ml of saturated aqueous Na₂SO₃ solution and 120 ml of water. After separation of the phases, washing of the organic phases with 80 ml of water and 80 ml of saturated NaCl solution, and removal of the solvent under reduced pressure, the product was obtained as a pale brown-red oil that solidified on cooling: yield 50.7 g (88.6% of theory).

¹H-NMR (CDCl₃, 400 MHz) δ (ppm)=7.41 (s, 2H), 4.76 (br s, 2H).

### 2,6-Dichloro-4-[1,2,2,2-tetrafluoro-1-(trifluoromethyl)ethyl]aniline

23.1 g (78.3 mol, 1.0 eq) of 2-chloro-4-[1,2,2,2-tetrafluoro-1-(trifluoromethyl)ethyl]aniline (III-2) as a solution in 100 ml of tert-butyl methyl ether from step (1) (Example (III-2c)) was metered into a suspension of 6.4 g (27.4 mmol, 0.35 eq) of 1,3,5-trichloro-1,3,5-triazine-2,4,6-trione (TCCA) in 50 ml of tert-butyl methyl ether at 0-5° C. over the course of 2 h. The reaction was warmed to 20-25° C. over the course of 2.5 h and stirred at this temperature for 1 h. The resultant solids were filtered off, and the clear solution was admixed with 20 ml of saturated aqueous Na₂SO₃ solution and 60 ml of water. After separation of the phases, washing of the organic phases with 40 ml of water and 40 ml of saturated NaCl solution, and removal of the solvent under reduced pressure, the product was obtained as a reddish-brown oil: yield 20.9 g (67.7% of theory).

¹H-NMR (CDCl₃, 400 MHz) δ (ppm)=7.41 (s, 2H), 4.76 (br s, 2H).

### 2,6-Dichloro-4-[1,2,2,2-tetrafluoro-1-(trifluoromethyl)ethyl]aniline (I-1f)

To 8.8 g (29.9 mmol, 1.0 eq) of 2-chloro-4-[1,2,2,2-tetrafluoro-1-(trifluoromethyl)ethyl]aniline (III-2) as a solution in 30 ml of ethyl acetate from step (1) (Example (III-2b)) were added, at 0-5° C., 0.15 g (1.5 mmol, 0.05 eq) of 96% by weight H₂SO₄ and then, in portions over the course of 1 h, 3.16 g (15.7 mmol, 0.53 eq) of 1,3-dichloro-5,5-dimethylhydantoin (DCDMH). The ice bath was removed and the reaction was stirred at room temperature for 2 h. Thereafter, the slightly cloudy solution was admixed with 10 ml of saturated aqueous Na₂SO₃ solution and 30 ml of water. After separation of the phases, dilution of the organic phase with 50 ml of ethyl acetate and subsequent washing of the organic phase with 30 ml of water and removal of the solvent under reduced pressure, the product was obtained as a beige-orange solid: yield 9.7 g (98% of theory).

¹H-NMR (CDCl₃, 400 MHz) δ (ppm)=7.41 (s, 2H), 4.76 (br s, 2H).

### 2,6-Dichloro-4-[1,2,2,2-tetrafluoro-1-(trifluoromethyl)ethyl]aniline (I-1g)

To 20.0 g (33.9 mmol, 1.0 eq) of 2-chloro-4-[1,2,2,2-tetrafluoro-1-(trifluoromethyl)ethyl]aniline (III-2) as a solution in 40 ml of ethyl acetate from step (1) (Example (III-2a)) was added 4.86 g (35.6 mmol, 1.05 eq) of N-chlorosuccinimide (NCS) in one portion at room temperature. This was followed by heating to 50° C. and stirring at this temperature for 3 h. Thereafter, the slightly cloudy solution was admixed with 10 ml of saturated aqueous Na₂SO₃ solution and 30 ml of water. After dilution of the organic phase with 40 ml of ethyl acetate, subsequent separation of the phases and removal of the solvent under reduced pressure, the product was obtained as a beige-orange solid: yield 10.4 g (93% of theory).

¹H-NMR (CDCl₃, 400 MHz) δ (ppm)=7.41 (s, 2H), 4.76 (br s, 2H).

### 2-Bromo-4-[1,2,2,2-tetrafluoro-1-(trifluoromethyl)ethyl]-6-(trifluoromethoxy)aniline (I-2)

To a solution of 234.6 g (0.68 mol, 1.0 eq) of 4-[1,2,2,2-tetrafluoro-1-(trifluoromethyl)ethyl]-2-(trifluoromethoxy)aniline (III-3) as a solution in 360 ml of ethyl acetate and 400 ml of n-heptane from step (1) (Example (III-3b)), after addition of 200 ml of water, was added a solution of 119.0 g (0.75 mol, 1.1 eq) of bromine in 40 ml of ethyl acetate at 25-30° C. over the course of 1 h. Over the entire metering time, the pH was set at 6-8 by addition of aqueous 53% by weight K₂CO₃ solution. By means of HPLC^{a}), complete conversion to the desired product was detected. The phases were separated, the organic phase was washed with 400 ml of aqueous 10% by weight sodium thiosulfate solution and dried, and the solvent was removed under reduced pressure at 40° C. The product was obtained as a dark red oil. yield 248.0 g (86% of theory).

¹H-NMR (CDCl₃, 400 MHz) δ (ppm)=7.59 (s, 1H), 7.34 (s, 1H), 4.65 (br s, 2H).

### 2-Chloro-4-[1,2,2,2-tetrafluoro-1-(trifluoromethyl)ethyl]-6-(trifluoromethyl)aniline (I-3)

To 4.0 g (12.1 mmol, 1.0 eq) of 4-[1,2,2,2-tetrafluoro-1-(trifluoromethyl)ethyl]-2-(trifluoromethyl)aniline (III-4) as a solution in 10 ml of ethyl acetate from step (1) was added a solution of 0.99 g (4.3 mmol, 0.35 eq) of 1,3,5-trichloro-1,3,5-triazine-2,4,6-trione (TCCA) in 5 ml of ethyl acetate at 0-5° C. over the course of 2 h. The reaction was warmed to 20-25° C. over the course of 2.5 h and stirred at this temperature for 1 h. The resultant solids were filtered off, and the clear solution was admixed with 10 ml of saturated aqueous Na₂SO₃ solution and 10 ml of water. After separation of the phases, washing of the organic phases with 15 ml of water and 15 ml of saturated NaCl solution, and removal of the solvent under reduced pressure, the product was obtained as a pale yellow oil: yield 3.16 g (71% of theory).

¹H-NMR (DMSO-d₆, 400 MHz) δ (ppm)=7.72 (br s, 1H), 7.46 (br s, 1H), 6.56 (br s, 2H).

### 2-Bromo-4-[1,2,2,2-tetrafluoro-1-(trifluoromethyl)ethyl]-6-(trifluoromethyl)aniline (I-4a)

20.0 g (60.8 mmol, 1.0 eq) of 4-[1,2,2,2-tetrafluoro-1-(trifluoromethyl)ethyl]-2-(trifluoromethyl)aniline (III-4) as a solution in 40 ml of ethyl acetate from step (1) was metered into a suspension of 9.3 g (31.9 mmol, 0.53 eq) of 1,3-dibromo-5,5-dimethylhydantoin (DBDMH) and 0.16 g (1.52 mmol, 0.025 eq) of 98% by weight H₂SO₄ in 100 ml of ethyl acetate at 20-25° C. over the course of 1 h. The reaction was stirred at this temperature for another 30 min. After addition of 25 ml of saturated aqueous Na₂SO₃ solution and 75 ml of water, the phases were separated, and the organic phase was diluted with 100 ml of n-heptane and washed again with 100 ml of water. Removal of the solvent under reduced pressure gave the product as a reddish oil: yield 20.4 g (82% of theory).

¹H-NMR (DMSO-d₆, 400 MHz) δ (ppm)=7.86 (br s, 1H), 7.50 (br s, 1H), 6.43 (br s, 2H).

### 2-Bromo-4-[1,2,2,2-tetrafluoro-1-(trifluoromethyl)ethyl]-6-(trifluoromethyl)aniline (I-4b)

4.0 g (12.1 mmol, 1.0 eq) of 4-[1,2,2,2-tetrafluoro-1-(trifluoromethyl)ethyl]-2-(trifluoromethyl)aniline (III-4) as a solution in 10 ml of ethyl acetate from step (1) was metered into a suspension of 1.9 g (6.4 mmol, 0.53 eq) of 1,3-dibromo-5,5-dimethylhydantoin (DBDMH) in 20 ml of ethyl acetate at 20-25° C. over the course of 1 h. The reaction was stirred at this temperature for another 30 min. After addition of 5 ml of saturated aqueous Na₂SO₃ solution, 15 ml of water and 25 ml of n-heptane, the phases were separated and the organic phase was diluted with 15 ml of water and 15 ml of saturated aqueous NaCl solution. Removal of the solvent under reduced pressure gave the product as an orange oil: yield 4.0 g (80% of theory).

¹H-NMR (DMSO-d₆, 400 MHz) δ (ppm)=7.86 (br s, 1H), 7.50 (br s, 1H), 6.43 (br s, 2H).

### 2-Bromo-4-[1,2,2,2-tetrafluoro-1-(trifluoromethyl)ethyl]-6-(trifluoromethyl)aniline (I-4c)

4.0 g (12.1 mmol, 1.0 eq) of 4-[1,2,2,2-tetrafluoro-1-(trifluoromethyl)ethyl]-2-(trifluoromethyl)aniline (III-4) as a solution in 10 ml of ethyl acetate from step (1) was metered into a suspension of 2.3 g (12.7 mmol, 1.05 eq) of N-bromosuccinimide (NBS) in 20 ml of ethyl acetate at 20-25° C. over the course of 1 h. The reaction was stirred at this temperature for another 60 min. After addition of 5 ml of saturated aqueous Na₂SO₃ solution, 15 ml of water and 25 ml of n-heptane, the phases were separated and the organic phase was diluted with 15 ml of water and 15 ml of saturated aqueous NaCl solution. Removal of the solvent under reduced pressure gave the product as an orange oil: yield 4.0 g (81% of theory).

¹H-NMR (DMSO-d₆, 400 MHz) δ (ppm)=7.86 (br s, 1H), 7.50 (br s, 1H), 6.43 (br s, 2H).

The following 4-perfluoroalkylanilines of the general formula (I) were preparable analogously to example (I-1d):

### 2-Chloro-4-[1,2,2,2-tetrafluoro-1-(trifluoromethyl)ethyl]-6-(trifluoromethoxy)aniline (I-5)

¹H-NMR (CDCl₃, 400 MHz) δ (ppm)=7.45 (s, 1H), 7.30 (s, 1H), 4.59 (s, 2H).

### 2-Chloro-6-ethyl-4-[1,2,2,2-tetrafluoro-1-(trifluoromethyl)ethyl]aniline (I-6)

¹H-NMR (CDCl₃, 400 MHz) δ (ppm)=7.43 s, 1H), 7.17 (s, 1H), 2.54 (q, J=7.5 Hz, 2H), 1.28 (t, J=7.5 Hz, 3H).

1. A process for preparing a compound of formula (I) 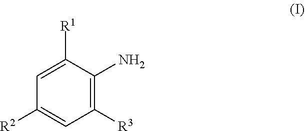
wherein
R¹ is chlorine or bromine,
R² is C₁-C₄-haloalkyl, and
R³ is cyano, halogen, optionally halogen- or CN-substituted C₁-C₄-alkyl or optionally halogen-substituted C₁-C₄-alkoxy,
from a compound of formula (II) 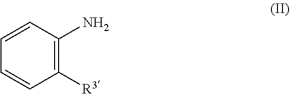
wherein R^{3′} is hydrogen, cyano, halogen, optionally halogen- or CN-substituted C₁-C₄-alkyl or optionally halogen-substituted C₁-C₄-alkoxy,
comprising the following steps (1) and (2):
(1) reacting a compound of formula (II) with compounds of a compound of formula R²—Y
wherein Y is iodine or bromine, to give a compound of formula (III) 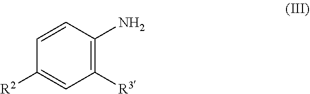
wherein R² and R^{3′} have the definitions given above; and
(2) chlorinating or brominating the compound of formula (III) with a chlorinating or brominating agent to give a compound of formula (I)
wherein the compound of formula (III) is not isolated from the reaction mixture from step (1) prior to step (2) and wherein an organic solvent is used in step (1) and no organic solvent is actively removed after step (1).

2. The process according to claim 1, wherein the compound of formula (III) from step (1) is used in step (2) directly as a solution in the organic solvent from step (1).

3. The process according to claim 1, wherein step (1) and step (2) are effected in the same reaction vessel.

4. The process according to claim 1, wherein the chlorinating or brominating agent in step (2) is selected from chlorine, bromine, N-chlorosuccinimide (NCS), N-bromosuccinimide (NBS), 1,3-dichloro-5,5-dimethylhydantoin (DCDMH), 1,3-dibromo-5,5-dimethylhydantoin (DBDMH), 1,3,5-trichloro-1,3,5-triazine-2,4,6-trione, 1,3,5-tribromo-1,3,5-triazine-2,4,6-trione or 1,3-dibromo-1,3,5-triazine-2,4,6-trione.

5. The process according to claim 1, wherein the organic solvent is used in step (1) is selected from the group consisting of acetonitrile, methyl acetate, ethyl acetate, isopropyl acetate, tert-butyl methyl ether, cyclopentyl methyl ether, THF and methyl-THF.

6. The process according to claim 1, wherein the compound of an organic solvent is used in step (2) and is selected from the group consisting of ethyl acetate, isopropyl acetate, tert-butyl methyl ether, THF, 2-methyl-THF, cyclopentyl methyl ether and acetonitrile.

7. The process according to claim 1, wherein the same organic solvent is used in step (1) and step (2).

8. The process according to claim 7, wherein the solvent is selected from ethyl acetate, isopropyl acetate, tert-butyl methyl ether, cyclopentyl methyl ether, THF, methyl-THF and acetonitrile.

9. The process according to claim 1, wherein R² is fluorine-substituted C₁-C₄-alkyl.

10. The process according to claim 1, wherein R² is perfluoro-C₁-C₃-alkyl.

11. The process according to claim 1, wherein R³ is Cl, Br, C₁-C₃-alkyl or fluorine-substituted C₁-C₃-alkyl, C₁-C₃-alkoxy or fluorine-substituted C₁-C₃-alkoxy, and R^{3′} is hydrogen, Cl, Br, C₁-C₃-alkyl or fluorine-substituted C₁-C₃-alkyl, C₁-C₃-alkoxy or fluorine-substituted C₁-C₃-alkoxy.

12. The process according to claim 1, wherein:
R¹ is chlorine or bromine,
R² is heptafluoroisopropyl,
R³ is chlorine, trifluoromethyl, trifluoromethoxy or difluoromethoxy, and
R^{3′} is hydrogen, chlorine, trifluoromethyl, trifluoromethoxy or difluoromethoxy.
